(19) United States
(12) Patent Application Publication
(10) Pub. No.: US 2009/0304610 A1
(43) Pub. Date: Dec. 10, 2009

(54) TANNIN POLYMERS, PROCESSES OF PREPARATION AND USE THEREOF

(76) Inventor: Carlo Ghisalberti, Sao Paulo (BR)

Correspondence Address: HESS PATENT LAW FIRM, P.C., 9 MIRAMAR LANE, STAMFORD, CT, 06902, US

(21) Appl. No.: 12/094,994

(22) PCT Filed: Nov. 29, 2005
(86) PCT No.: PCT/IB2005/003581

§ 371 Date: Aug. 7, 2008

(51) Int. Cl. A61K 8/84; C08G 65/44; C08K 3/22; A61Q 17/04; D06P 3/32; C09B 67/08; A61Q 1/04; A61Q 1/02; A61Q 1/10; C08G 63/06

(52) U.S. Cl. 424/59; 528/208; 524/413; 424/63; 8/436; 8/637.1; 524/432; 524/430; 524/403; 524/407; 424/64

(57) ABSTRACT

The invention concerns synthetic polymers obtainable by oxidative polymerization of vegetal tannins, alone or in combination with eumelanin and/or pyrocatechic precursors, method of preparation and uses thereof in the cosmetic and dermato-logic fields; complexes of said polymers with metals and their use in dyeing processes are also disclosed.

### FIELD OF INVENTION

The present invention refers to synthetic polymers, herewith named “tanmelanins”, obtainable by oxidative polymerization of vegetal tannins, alone or in combination with eumelanin and/or pyrocatechic precursors, as well as methods of preparation thereof by oxidative, chemical Or enzymatic polymerization.

The present invention also refers to topical cosmetic and dermatologic composition comprising tanmelanins with the function of solar filter, bacteriostatic, chelating and antioxidant for the application to skin and mucosa.

The present invention also refers to metal-tanmelanin complexes obtained by the partial ionic exchange with polyvalent cations of the alkaline salts of tanmelanins as obtained upon the process of preparation by oxidative polymerization.

The present invention furthermore refers to a method for dyeing a substrate comprising the step of applying to said substrate the tanmelanin in alkaline or semi-acidic form or as metal-tanmelanin complexes.

### BACKGROUND

Tannins and melanin represent two large classes of biopolymeric polyphenols with a role of endogenous protection and are widely diffused in nature, the former in the plants, the latter produced both by animals and by plants and fungi.

A broad classification of melanin be made on the basis of the starting monomers (the melanin precursors), major classes being: eumelanins, pheomelanins and allomelanins.

Eumelanins are brown-black nitrogenous pigments produced by the rearrangement and oxidation of L-dopa and its metabolites. A type of eumelanins are the “oxymelanins” exhibiting tetrahydroxy indoles units within their polymeric structure. Other eumelanins are the “neuromelanin” from dopamine (which is produced L-dopa carboxylase) and its metabolite, 3,4-dihydroxycarboxylic acid; as well as by neural catecholamines and tetrahydroxyisoquinolines, e.g. occurring in the brain substantia nigra.

Pheomelanins are yellow-reddish melanins containing both nitrogen and sulfur atoms, which arise from the rearrangement and polymerization of cysteinyl-dopa, the condensation product of L-dopa and glutathione with further enzymatic hydrolysis. The pheomelanins generally result from the oxidative condensation of 1,4-benzothiazines. Further pheomelanins are “trichochromes” e.g. containing bi-benzothiazinic chromophore (—S≡C—C═N—); or the condensed 1,4-benzothiazinalanines (e.g. in tricochrome C).

Allomelanins typically occur in the plant kingdom and may be further classified as “vegetal melanins” when resulting from the polymerization of plant polyphenols; and “fungi melanins” (alias “micomelanins”) when containing naphthoic groups.

Tannins are vegetal biopolymer of molecular weight comprised between 1000 and 6000 dalton and up to 30000 dalton comprising phenolic moieties, optionally oxidated to semiquinones and quinones, and a variety of chelating centers having proximal and/or vicinals phenolic or carbonyls groups.

The tannin are both hydrolizable and non-hydrolyzable molecules. Based on the classification of Khanbabaee and van Ree (Nat. Prod. Rep., 2001, 18:641-649) the former are generally represented by gallotannins, ellagitannins and complex tannins, the latter by condensed tannins.

In our previous patent application we have described melanin formed by oxidative polymerization of simple polyphenols and eumelanin precursors (WO01/018125) as well as melanin formed by oxidative polymerization of dihydroxynaphthtoic and eumelanin precursors (WO02/02066).

### SUMMARY OF THE INVENTION

We have now found out that tannins represent ideal precursors to prepare novel melanin-like polymers, thereby obtained by oxidative polymerization of said tannin precursors, which represent a new tool among the biopolymers available for industrial applications.

Therefore, the present invention refers to synthetic polymers, herewith after also “tanmelanins”, obtainable by oxidative, chemical or enzymatic polymerization of tannin precursors of vegetal origin and optionally by copolymerization with eumelanin and/or pyrocatechic precursors.

The invention also refers to the method of preparation of said tanmelanins by oxidative polymerization, preferably in aqueous alkaline solution.

The tanmelanins are also suitable for the manufacturing of topical cosmetic and dermatologic compositions having sun filter, bacteriostatic, chelating and antioxidant properties, as well as in decorative (make-up) and in sun-protecting formulations either in form of soluble tanmelanins or as pigments formed by deposition of tanmelanins on micronized pigments.

Also, novel metal-tanmelanin complexes can be produced by ionic exchange of the tanmelanins alkaline salts with polyvalent cations.

Further applications of tanmelanin salts and metal complexes have been found in the industrial dyeing, i.e. in printing textile fibres such as wool, silk, cotton, linen, hemp, etc., and in dye-tanning of bovine, ovine-caprine, and exotic leather.

### DETAILED DESCRIPTION OF THE INVENTION

According to one aspect, the present invention refers to synthetic polymers herewith after also called “tanmelanins”, their soluble salt, and cationic complexes thereof, herewith after “metal-tanmelanin complexes”, constituted by tannin monomers (a), (b) and optionally eumelanic (c) and/or pytocatechic monomers (d) according to the formula (1):

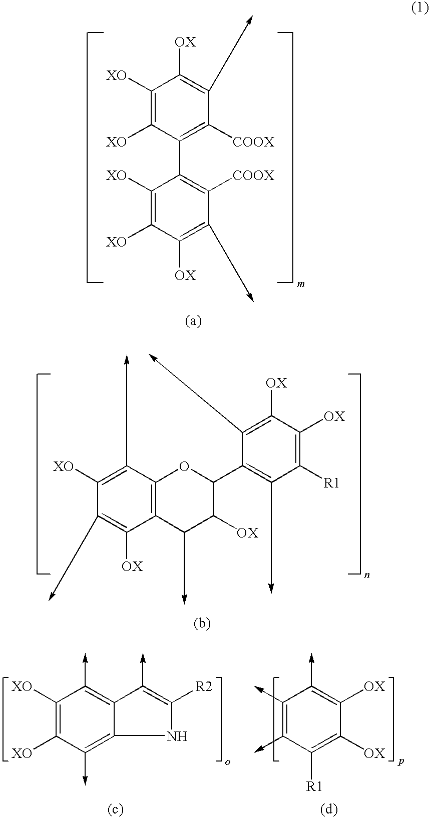

wherein:

→ is a possible intramolecular link between the monomeric units;

m and n are, each independently, a integer between 5 and 30;

o and n are, each independently, a integer between 0 and 30;

R₁ is —OX or —H;

R₂ is —COOX or —H;

each X is independently —H, -G, -M, or a further intramolecular link;

G is a glucosidic, glycosidic or polyolic residue;

M is M₁, 1/y·M₂^{y+}, or 1/z·M₃^{z+};

M₁, is a monovalent cation;

M₂ is a non-chromophoric multivalent cation;

M₃ is a chromophoric multivalent cation;

y and z are integer from 2 to 4 and represents the valence of M₂ and M₃.

According to the present invention, when X is H the corresponding —OH group may be in a radicalic (—O.) or quinonic (═O) form.

The monovalent cation M₁ is preferably an alkali metal cation (especially Li, Na or/and K) or an ammonium cation (e.g. NH₃ or ammonium mono-, di- or tri-substituted with C₁₋₂-alkyl and/or C₂₋₃-hydroxyalkyl), alkali metal cations being preferred.

The non-chromophoric multivalent cation M₂ is any colourless transition metal suitable for metal complex formation, preferably Zn, Mg, Ca, Ba, Al, or Ti.

The chromophoric multivalent cation M₃ is any coloured transition metal suitable for metal complex formation, preferably Cr, Cu, Co, Fe, Mn, V, or Zr.

According to the present invention the expressions 1/y·M₂^{y+} and 1/z·M₃^{z+} mean that only one valence of the multivalent cations is joined to the oxygen one —OX group. As a consequence, one multivalent cation is linked to y or z —OX groups.

The polymer of the invention may advantageously also be in partial salified form, i.e. where some of the M are salt-forming cations and some are hydrogen (or hydronium).

M may be any cation as is formed in the synthesis of the metal complex, e.g. as indicated above for M, and depends thus also on M₂ or M₃ and further also on the complex forming reaction conditions (namely the pH and the employed solvent), or a cation introduced by ion exchange; in the free acid form it is indicated as a hydronium ion.

Preferably the tanmelanins of the invention contain different monomer units (a), (b), (c) and (d), whose ratio are preferably as follows:

(m+n): (O) comprised between 10:0 and 2:8;

(m+n): (p) comprised between 10:0 and 4:6; and

(m+n): (o+p) comprised between 10:0 and 3:7.

According to an embodiment, the tanmelanins of the invention contain eumelanin units (c), with (m+n): (o) comprised between 9:1 and 1:1.

According to a further embodiment, the tanmelanins of the invention contain pyrocatechic units (d), with (m+n): (p) comprised between 9:1 and 1:1.

According to a further embodiment the tanmelanins of the invention contain all monomers (a), (b), (c) and (d), with (m+n): (o+p) comprised between 8:2 and 1:1.

The tanmelanins of the invention are obtainable by oxidative polymerization of tannin precursors and, optionally, eumelanin and/or pyrocatechic precursors.

The oxidative polymerization of vegetal tannin precursors, alone or by copolymerization with eumelanin and/or pyrocatechic precursors constitutes a further subject-matter of the present invention.

Exemplary tannin precursors of type (a) and (b) can be selected among the following:
1. Ellagitannin precursors, illustrated by formula (2):

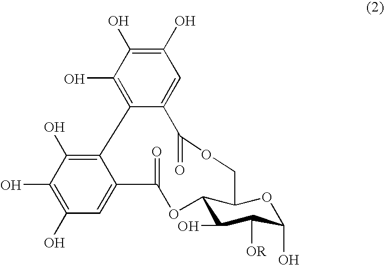

2. The condensed tannin precursors have condensed catechic structures comprising catechin, epicatechin and leucocyanidin with C—C bonds, therefore they are generally not cleavable under the classic hydrolytic conditions. An example of condensed tannin precursors is illustrated in the formula (3):

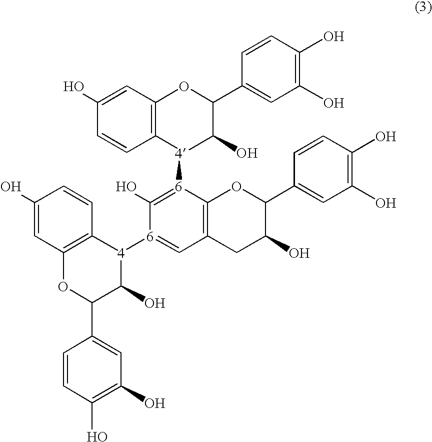

3. Complex tannin precursors are hybrid of 1) and 2) and are therefore constituted of gallic and ellagic acid glycosides and condensed catechins, which are therefore only partially hydrolyzables. The simpler terms are punicalin and acutissimin A.

An example of complex tannin precursor is illustrated in the formula (4):

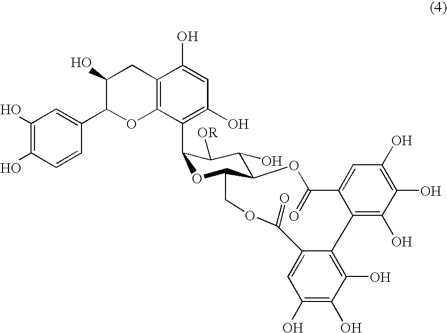

Other suitable tannin precursors may have a glycosidic and/or polyolic group (“G”) different from those indicated in the above mentioned formulae, which are exemplary illustrated by the formulae 5-38 herewith after:

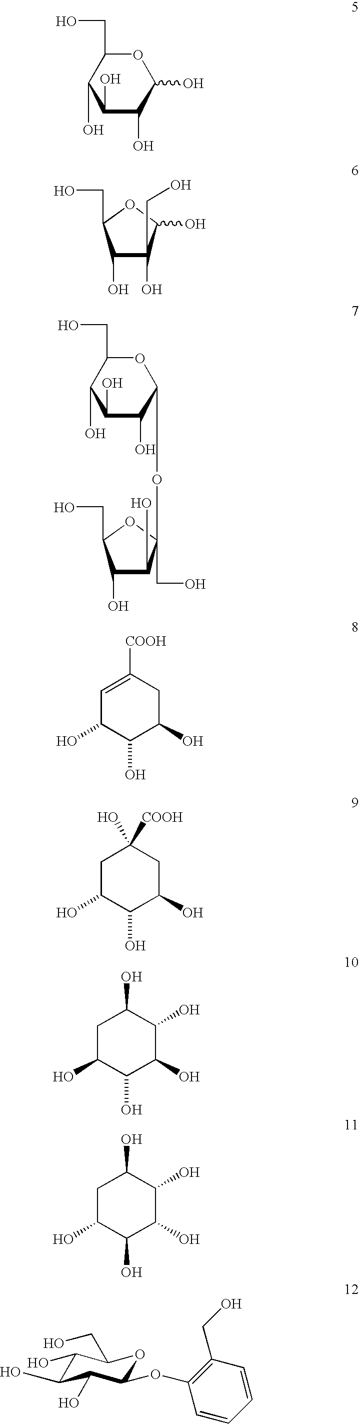

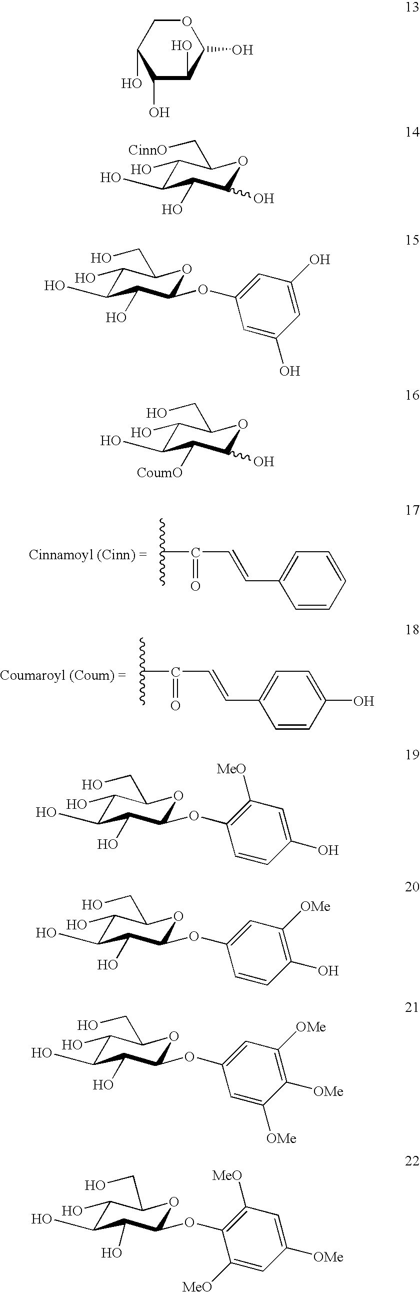

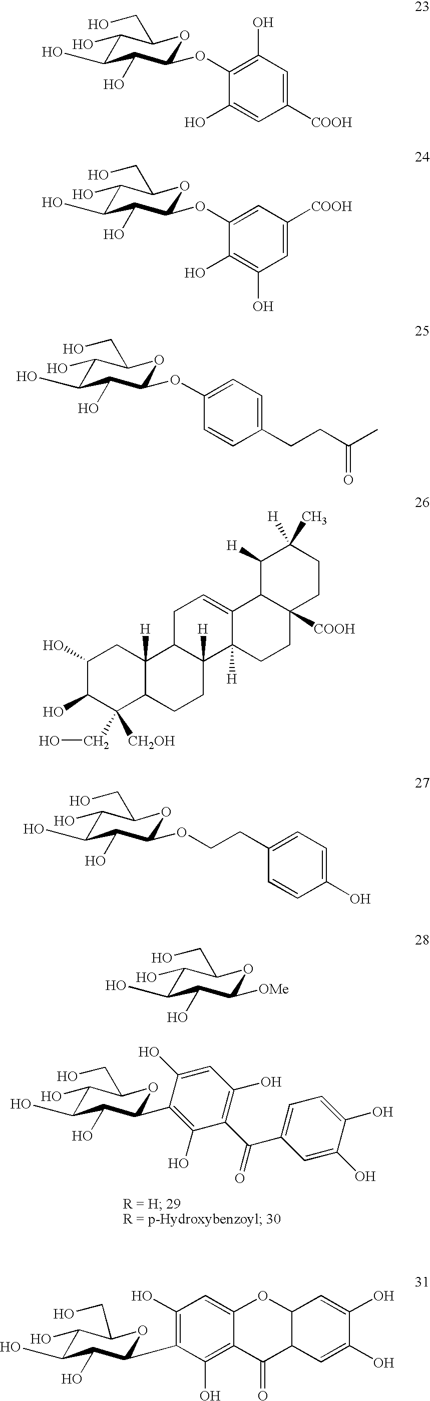

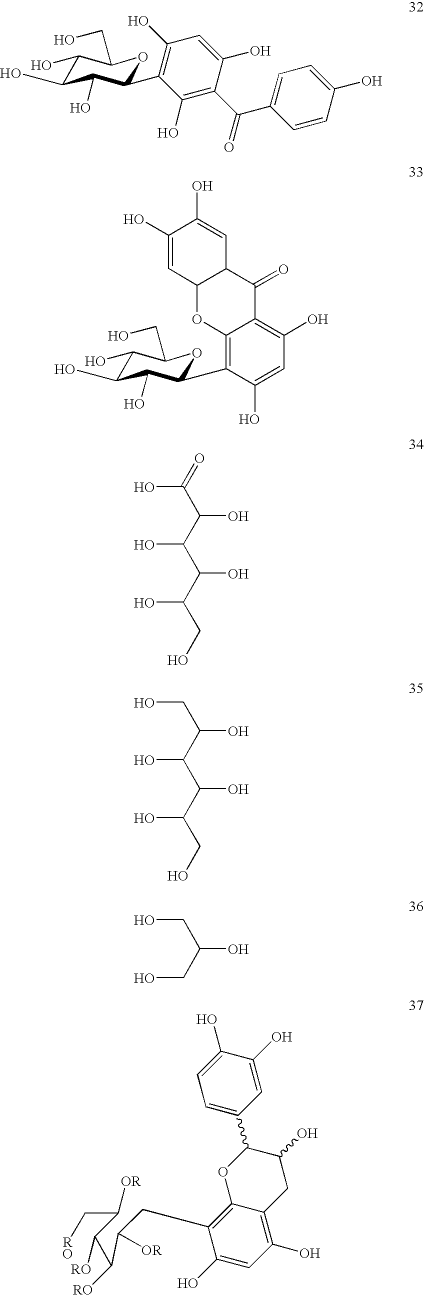

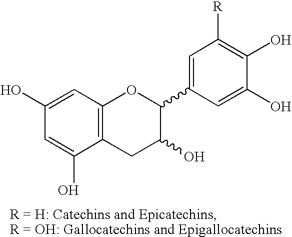

The glycosidic-polyolic residues linked to the tannin precursors according to the invention are generally cleaved during the oxidative polymerization in alkaline conditions.

Nevertheless, some glycosidic-polyolic residues may persist in the tanmelanins. In such case, some of the —OH and —COOH groups of the polymers of formula (1) are esterified (X=G).

Preferably the tannin precursors are selected among those of formulae 1-3, to form polymers comprising units (a) and (b).

The tannin precursors suitable for the preparation of the tanmelanins of the invention are contained in a variety of vegetal genera, particularly from Rhus semialata e Quercus infectoria (chinese or turk galls), from Caesalpinia spinosa (Tara); condensed tannins from Schinopsis spp. (quebracho), from Acacia catechu (catechu) or Acacia mollisima (mimosa), from Rhizophora (mangrovia) or from Eucalyptus o Uncaria gambier; whilst mixed tannins are obtained from Castanea sativa (castagno) or Rhus semialata (sumac).

Suitable tannin precursors for the preparation of tanmelanins of the invention preferably have a minimum titre of 50% and/or further purified fractions, which are either commercially available or are obtainable by known fractionation techniques.

When the tanmelanins of formula (1) contain monomer (c), the oxidative polymerization method shall comprise the presence of eumelanin precursors.

Preferred eumelanin precursor is L-dopa of formula 1′, preferably of vegetal origin (e.g. from Mucuna p) which, during the oxidative polymerization produce 5,6-dioxyindol-4′-carboxylic acid and 5,6-dioxyindol, R²═COOH and R²=H respectively in formula 2′:

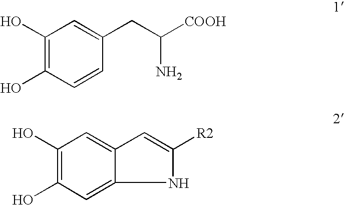

When the tanmelanins of formula (1) contain monomers (d), the oxidative polymerization method shall comprise the presence of pyrocatechic precursors.

A pyrocatechic precursor include pyrocatechin (1″) and pyrogallol (2″), pyrocatechin of formula 1″ being preferred:

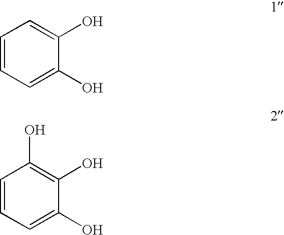

The oxidative polymerization of the aforementioned precursors affording polymers of formula (1) may be carried out by oxidative polymerization, preferably in alkaline solution.

The tanmelanins of the present invention are primarily characterized by distinctive and reproducible physico-chemical features such as the molecular weight, the colour and their spectroscopic features, thanks to the control of the starting monomeric composition and of the polymerization conditions.

A preferred method of producing the tanmelanins is the chemical method, i.e. bubbling air or oxygen through an alkaline aqueous solution of the monomers at pH 10 or above (e.g. by NaOH 1N or ammonia 2-3 N) during 8 to 48 hours (preferably 24 hours), at a temperature ranging from 10 to 90° C., preferably from 20 to 40° C. The alkaline aqueous solution may comprise catalytic amounts of pro-oxidant metals (M₃), such as Cu⁺⁺, Fe⁺⁺, Ni⁺⁺, or Co⁺⁺ solutions, for example at 5 to 20 mM concentration.

According to a preferred embodiment, a method of oxidative polymerization for the production of a polymer according to the invention comprises:
a) formation of an alkaline solution of the precursors by the addition of the precursors at 1 and 20% w/v to an aqueous solution containing an amount of alkali at least stoichiometric with regards to the —COOH and —OH groups;
b) dispersion of air, or a gas mixture comprising oxygen, into the solution (a) at a temperature between 0 and 80° C. for a period comprised between 2 and 48 hours to afford tanmelanin in the alkaline salified form, i.e. with prevailing X=M₁;
   optionally followed by:
c) partial or complete neutralization by addition of mineral acid (e.g. HCl, H₂SO₄) or water-soluble organic acid (e.g. acetic, lactic acid) to afford tanmelanin in the acidic form, i.e. with prevailing X═H;

In the afore polymerization, step (b) may be carried out in the presence of further oxidizing agents, such as hydrogen peroxide (H₂O₂), Na₂S₂O₈, NaClO, HI, or Mg(ClO₄)₂ being preferred to afford tanmelanin of pale colour.

According to another aspect, the present invention refers to tanmelanins further comprising secondary co-monomers such as anthocyanin and flavonoids.

The anthocyanins appear in the petals of flowers, in the leaves of many plants and in the fruit of coloured fruits and vegetables, in skins (egg plant, apple), in the fruit body (cherry, blackberry), or in peel and pulp of grape. The chemistry of these materials based on 2-phenylbenzopyrylium (flavylium) substituted with OH or CH₃O groups include: pelargonidin, cyanidin, delphinidin, petunidin, peonidin, malvidin, which may also be mono, di and tri-saccharides, and position 3 may be acylated, e.g. with p-coumaric acid.

Flavonoides include a large structural variety of compounds such as: flavandiols (e.g. dihydroquercetin), flavanonols (e.g. miricetin), flavones (e.g. luteolin), flavonols (e.g. quempferol, morin and quercetin), flavanones (e.g. esperetin), flavones (e.g. naringenin), flavononols (fisetin) and flavanonols (fustine), including glycosides and esters thereof.

Such co-monomers are introduced by oxidative co-polymerization of the tanmelanin precursors as above indicated and the anthocyanin and flavonoid precursors.

The polymers obtainable and/or obtained with the method of the invention constitute a further subject-matter of the invention.

The tanmelanins obtained with the method of invention are cross-linked 3-D macropolymers with a broad absorption spectra within the UV-visible range; moreover having strong chelating and free radical scavenger activities. These properties are particularly useful for application in the cosmetic and dermatologic fields.

Therefore, according to another aspect the present invention refers to topical composition comprising tanmelanins with the function of sun filter, bacteriostatic, chelating and antioxidant for the application to skin and mucosa.

So, it is a further subject-matter of the present invention to provide cosmetic compositions comprising the aforementioned tanmelanins in amount between 0.001 and 10% by weight, preferably between 0.01 and 1% by weight of the composition.

The cosmetic composition according to the invention may further comprise any cosmetically acceptable ingredients, for example those included in the INCI list drawn by the European Cosmetic Toiletry and Perfumery Association (COLIPA) and issued in 96/335/EC “Annex to Commission Decision of 8 May 1996” and further modifications.

Preferred cosmetic ingredient to be associated at the tanmelanins in fluid cosmetic formulations are the surfactants, thus including anionic, cationic, nonionic and zwitterionic surfactants, either with medium-low molecular weight or as oligopolymer and polymer containing a polarized moiety within a predominantly hydrophobic structure.

More preferably, the tanmelanins in solution are partially neutralized and premixed with one or more surfactant (e.g. soybean lecithin and mono- di-glycerides) prior the mixing with the rest of the formulation, to produce a stable colour, an homogeneous appearance and enhancing stability and shelf life of the cosmetic composition containing thereof.

Particularly preferred are cosmetic and dermatologic compositions intended for topical use, which can be liquid, paste or solid form, for example as water-in-oil creams, oil-in-water creams and lotions, aerosol foam creams, gels, oils, marking pencils, powders, sprays and alcoholic-aqueous lotions, pastes, protective lipstick or fat-free gels.

The tanmelanins can be used in cosmetic composition either in solution or in solid form.

The tanmelanins in solid form can be obtained by the addition of water-soluble organic solvents (e.g. acetone, ethanol, methanol), which decrease the solubility of tanmelanins as the partially/fully neutralized solution as obtained in step (c), i.e. with prevailing X═H.

The tanmelanins of the present invention can be obtained in solid form as non-staining pigments by co-precipitation with of the tanmelanin in alkaline solution as obtained in step (b) by M₂ or M₃, preferably M₂ such as Zn, Mg, Ca, Ba, Al, and Ti on micronized pigments, preferably inorganic oxides and hydroxides including TiO₂, Ti(OH)₂, ZnO, Al₂O₃, AlOOH, SnO₂, CeO₄, ZrO₂, FeO, Fe₂O₃, Fe₂O₃.H₂O, FeₓO_{y}, Fe₃O₄; Cr₂O₃.nH₂O, Cr₂O₃, SnO, and MnO.

The pigment of invention include nacres and interference pigments such as mica, sericite and muscovite containing, or surface-treated with, said oxides and hydroxides.

The tanmelanins in solid form may be recovered from the aqueous suspension by filtration, sedimentation, liophylization, atomization, jet-milling or centrifugation.

Therefore, according to another aspect, the present invention refers to metal-tanmelanin pigments and method to prepare thereof.

The tanmelanin pigments and their use in cosmetic compositions for make-up composition (e.g. foundation, pressed powder, face powder, lipstick, eye shadow, eyebrow pencil, eye liner, mascara, anhydrous or hydrated emulsion, and paste) and sun protecting products constitute a further subject-matter of the present invention.

In a further embodiment, the present invention refers to metal-tanmelanin complexes obtained by the partial ionic exchange with polyvalent cations of the alkaline salts of tanmelanins as obtained upon the process of preparation by oxidative polymerization.

The metal-tanmelanin complex of the invention may advantageously also be in partial salified form, i.e. where some of the X are salt-forming cations M₂ or M₃ and some are monovalent cations (M₁) or hydrogen (or hydronium).

The metal-tanmelanin complex of the present invention are suitably obtained by reacting a tanmelanin in alkaline form (X=M₁) with a heavy metal donor, preferably a chromophoric multivalent cation, M₂, i.e. a coloured transition metal suitable for metal complex formation, in particular Cr, Cu, Co, Fe, Mn, V, and Zr.

Suitable heavy metal donors are, for example, freshly precipitated hydroxides or water-soluble inorganic salts of Co, Cr, Cu, Fe, Zr or Ni, in particular the chlorides, fluorides, nitrates or sulfates, for example CoSO₄, CrCl₃, Cu SO₄ or NiSO₄, herewith after also referred as “metallizing agents”. Chroming can also be effected with chromates in the presence of a reducing agent.

The tanmelanins are preferably reacted with the metallizing agents in the equivalent ratio 1:1 or 1:2 under the conditions normally employed for these metallizing reactions to afford metal-tanmelanin complexes which are soluble and can be used as dyes in industrial applications.

Upon completion of the required metallization reactions the obtained dyes or mixtures thereof may be isolated in a manner conventional per se, e.g. by salting-out or by acidification with a mineral acid or e.g. by evaporation, upon dialysis with a suitable membrane. If desired, the dye may, upon isolation or dialysis, be blended with suitable blending agents conventional per se, e.g. with alkali metal salts (Na₂CO₃, Na₂SO₄ or NaCl), with non-electrolyte blending agents (mainly urea or/and oligosaccharides, e.g. dextrin) or/and with anionic surfactants, in particular organic sulfonates, e.g. sulfonated castor oil, sulfosuccinates or lignin sulfonate. If a surfactant is employed, the weight ratio of the surfactant to the dye is advantageously in the range of 5:95 to 40:60. If desired, especially if the composition contains an anionic surfactant, it may be formulated with water as concentrated liquid dye compositions, preferably with a dry-substance content in the range of 10 to 70%, more preferably 20 to 50% by weight of the composition.

In a further embodiment, the present invention refers to a method for dyeing a substrate dyeable with anionic dyes comprising the step of applying to the substrate tanmelanin in alkaline or semi-acidic form or, preferably, in the form of metal-tanmelanin complexes.

The tanmelanins and the metal complex thereof are particularly suitable as dyeing agent, in printing textile fibres and particularly natural fibres such as wool, silk, cotton, linen, hemp, etc., and in dye-tanning leather such as bovine, ovine-caprine, and exotic leather.

The dyeing may be carried out in a manner known per se, suitably in an aqueous medium and under conventional temperature and pH conditions, e.g. in the temperature range of 20-80° C., preferably 25-70° C., milder temperature conditions of 25-40° C., being preferred for the achievement of deeper penetrations and for the dyeing of woolled skins and furs. The pH-values of the dye-bath may, in general, range broadly; mainly from pH 9 to 3; in general the dyeing may be advantageously started at higher pHs and concluded at lower pHs. Preferably the dyeing is carried out at pH>4, in particular in the range of 9 to 4 and for the conclusion of the dyeing process the pH value is lowered at 3-4 e.g. by addition of acetic or formic acid. The dye concentration may range broadly, up to the saturation degree of the substrate, or. up to 5% on wet weight of the substrate. The dyeing may be carried out in one or more stages, e.g. in two stages, optionally with insertion of charge reversal of the substrate by means of conventional cationic assistants. The dyeing may be carried out in the presence of a dyeing adjuvant; i.e. conventional non-ionic or anionic products, in particular hydrophilic surfactants, e.g. hydrophilic polysaccharide derivatives, polyoxyethylated alkyl phenols, ligno-sulfonates or sulfo-aromatic compounds.

The dyeing method of the present invention is characterised chiefly by a good penetrating and covering properties and good fastness to diffusion in leather. In the application to leather a good fastness to light, washing, water, perspiration, acid, alkali and rubbing is attained. The shades which can be obtained range from yellow to black.

The dyes according to the invention can be applied to and fixed on the substrates mentioned, especially the fiber materials mentioned, by the application techniques known for water-soluble, fiber-reactive dyes, in accordance with the use of the invention, for example by applying the compounds of formula (1) in dissolved form to the substrate or incorporating them thereinto and fixing them thereon or therein by heating or by the action of an alkaline agent or by both measures. Such dyeing and fixing processes are extensively described in the literature, for example in EP-0181585.

Thus the polymers of formula (I) are useful not only for the exhaust dyeing process but also for dyeing by the pad dyeing process, whereby the material is impregnated with aqueous solutions of the dyes according to the invention, which may contain electrolyte salt, and fixed on the material in the manner mentioned above. The compounds of formula (I) are particularly useful for cold pad-batch processes, whereby a dye is applied to the fiber material on a pad mangle together with alkali and is fixed by storing at room temperature for several hours. After fixing, the dyes obtained are thoroughly washed with cold and hot water, if appropriate in the presence of an agent which has a dispersing action and promotes the diffusion of unfixed portions.

The compounds of formula (I) are notable for high reactivity, good flexibility and good build-up capacity. They can therefore be used for exhaust dyeing at low dyeing temperatures and require only short steaming times in pad-steam processes. The fixation yields are high, and the unfixed portions are readily washed off with the difference between the degree of exhaustion and the degree of fixation being remarkably small, i.e., the loss through hydrolysis being very small.

The polymers of formula (I) are also particularly useful in textile printing processes, especially on cellulose fibres, such as cotton, but also for printing carboxamide-containing fibres, for example wool and silk, or blend fabrics containing wool or silk.

### EXAMPLES

### General Method A

### Synthetic Procedure by Oxidative Polymerization

A weighted amount of monomers is charged into a flat-bottom conical flask (2 l) and solubilized in 100 ml of NaOH 1 N. The flask, placed in a thermostatic bath at 24° C., is equipped with an air pump (Silent Air®, manufactured by Renn-Plax, Inc., Taiwan R.O.C.) connected by a flexible hose to a disperser, which is placed at the bottom of the flask. The air is forced through the disperser and finely divided throughout the alkaline solution, for a time from 8 to 48 hours (12 hours in the Examples to follow) at a temperature of 25-40° C. The water lost due to the evaporation caused by the stream of air was refilled from time to time in the reaction vessel.

The reaction mixture readily develops colours, as tanmelans absorb widely throughout the UV and visible spectra (e.g., 220 to 700 μm), the final colour of the melanin solution varying according to the monomer composition and, at less extent, to the reaction time.

The reaction end up with solutions of intense red-brown colours containing 10% tanmelanins as alkaline salts.

Absorbance of tanmelanins was measured by spectrophotometric absorbance of a solution obtained by dissolving 4 mg of synthetic vegetal melanin in 100 ml of 0.1 N NaOH. The UV visible absorption spectrum was recorded on a spectrophotometer between the wavelength of 200-700 nm in 1 cm path length cuvette.

Tanmelanin can be analysed by chromatographic (HPLC) method. A sample buffered at pH 9 with acetic acid is analysed on Lichrospher RP 18 equipped with precolumn of 25 mm packed with Perisorb A (Anspec Co. Inc., Ann Arbor, Mich., USA) and column packed with 10 cm 5-μm Lichrocart (Merck, Darmstat, Germany). The mobile phase is H₂O/CH₃OH/H₃PO₄-80% (100:1:0, 1 v/v/v) and the eluents are measured at 280 nm.

### Examples 1-8

### Tanmelanin Synthesis

The tanmelanins in alkalin form were prepared by applying for 24 hours the condition of oxidative polymerization according to the General Method A of the precursors in the proportion illustrated by the Table I.

TABLE I

| Ex. | Tara | Quebracho | | | Structure of the |
|---|---|---|---|---|---|
| N^{o}. | tannin (1) | tannin (2) | L-dopa | Pyro_catechin | resulting polymer |
| 1 | 100 g | — | — | — | m > 5 |
| 2 | — | 100 g | — | — | n > 10 |
| 3 | 50 g | 50 g | — | — | m:n ~4:3 |
| 4 | — | 66.6 g | 33.3 g | — | m:o ~1:1 |
| 5 | — | 43.3 g | 36.7 g | 20 g | m:n:o ~1:4:4 |
| 6 | — | 75 g | 15 g | 10 g | m:n:o ~1:1:1 |
| 7 | — | 80 g | — | 20 | n:p ~1:1 |
| 8 | 75 g | — | — | 25 g | m:p ~1:1 |

(1) Typical composition: galloellagitannin 60%, gallic acid 9.5%.
(2) Typical composition: (+)-mollisacacidin 2%, condensed tannin 58%.

### Example 9

### Inhibitory Activity in Metal-Catalysed and in Free Radical Induced Lipidic Peroxidation

The inhibition activity of the synthetic vegetal melanins has been carried out according to Visioli F. and Galli C. “Evaluating oxidation processes in relation to cardiovascular disease: a current review of oxidant/antioxidant methodology.” in Nutr. Metab. Cardiovasc. Dis. 7: 459-466 (1997).

Briefly, human LDL (d=1.021-1.063) were isolated per sequential ultracentrifugation from plasma obtained from healthy, normolipidemic volunteers. Before initiation of the experiments, LDL samples were desalted by size-exclusion chromatography and their protein content was determined according to Lowry O., J. Biol. Chem., 193, 265, 1951.

The tanmelanin of the Example 6 at 1, 5, 10 and 20 ppm were added and oxidation started by the addition of the free radical generator 2,2′-azo-bis(2-amidinopropane) dihydrochloride (AAPH) at concentrations of 5 mM. Incubation were carried out at 37° C. in a shaking bath and aliquots were withdrawn at different times for the analysis of the tiobarbituric acid reacting substance (TBARS) as oxidation marker, as shown in Table II.

The probes were thereby pre-incubated for 30 minutes at 37° C. with the tanmelanin of the Example 6 at 1, 5, 10 and 20 ppm, then copper sulfate (5 uM, final concentration) was added to start the oxidation, which was carried out at 37° C. in a shaking bath. At the end of the incubation period, samples were withdrawn for the quantization of the tiobarbituric acid reacting substances (TBARS). The tanmelanin inhibition in the metal-dependent oxidation was IC₅₀˜5 ppm and IC₈₀˜10 ppm; whilst the inhibition value in the free radical oxidation were IC₅₀-10 ppm and IC₇₀˜20 ppm.

### Applicative Example 1

### O/W Sun Cream (SPF 7)

| Phase | g/100 g | Ingredient |
|---|---|---|
| A | 12.00 | Glyceryl Stearate SE (Tegin) |
| | 1.00 | Ceteareth-6, Stearyl Alcohol (Cremophor ™ A 6) |
| | 1.00 | Ceteareth-25 |
| | 10.00 | Caprylic/Capric Triglyceride |
| | 10.00 | PPG-3 Myristyl Ether |
| | 3.00 | Octyl Triazone |
| B | 3.00 | Benzophenone-4 |
| | 3.00 | Tetrahydroxypropyl Ethylenediamine |
| | 0.50 | Tanmelanin of the Example 5 |
| | 5.00 | Glycerin |
| | q.s. | Preservative |
| | ad 100 | Aqua dem. |
| C | q.s. | Perfume |

### Applicative Example 2

### After-sun Milk

| Phase | g/100 g | Ingredient |
|---|---|---|
| A | 5.00 | Isopropyl Palmitate |
| | 4.00 | Cetearyl Isononanoate |
| | 4.00 | Soybean Oil |
| | 3.00 | Caprylic/capric Triglycerides |
| | 3.00 | Jojoba Oil |
| | 1.00 | Wheatgerm Oil |
| B | 0.30 | EDTA-sodium salt |
| | 3.00 | Glycerol |
| | 1.00 | Tanmelanin of the Example 7 |
| | 0.20 | Allantoin |
| | ad 100 | Aqua demineralized |
| | q.s. | Preservative |
| C | q.s. | Perfume |

### Applicative Example 3

### Pigment TiO₂/Ca-tanmelanin

Into a 1 litre beaker fitted with a mechanical stirrer were suspended 200 g of a TiO2 in 500 ml of water, then the slurry was warmed at 80-85° C. There was started the addition of 80 ml of Tanmelanin of the Example 6, thereby stirred for further 15 minutes at the same temperature. Thereafter, the slurry was slowly added with then 40 ml of CaCl₂ 1M and stirred for further 20 minutes. The slurry was then cooled to room temperature, filtered and the filtrate is washed with warm deionized water until soluble salts are removed, the resulting cake was dried at 10° C. and grinded, to afford a beige powdered product.

### Applicative Examples 4-6

### Pigments Iron oxides/Al-tanmelanin

By applying the method of Appl. Example 3 using 100 g of yellow, red and black iron oxides instead of TiO₂, and 3×7 ml of AlCl₃ 1M instead of CaCl₂, micronized pigments formed by iron oxides coated with tanmelanin were obtained. The yellow, red and black hues of the original pigments look intensified (slightly darkened) after the surface treatment with tanmelanin.

### Applicative Example 7

### Foundation Containing Tanmelanin-treated Pigments

| g/100 g | Ingrediente |
|---|---|
| 2.50 | Trietanolamine stearate |
| 0.40 | Glyceryl mono- and distearate |
| 1.90 | Magnesium silicate |
| 1.00 | Pigment of the Applicative Example 3 |
| 1.00 | Pigment of the Applicative Example 4 |
| 0.60 | Pigment of the Applicative Example 5 |
| 0.30 | Pigment of the Applicative Example 6 |
| 9.00 | Mix of PEG-6 and PEG-32 |
| 9.00 | Micronized nylon |
| 13.0 | Cyclomethicone |
| 5.00 | Propylen glycol |
| 4.50 | Glycerin |
| q.s. | Preservative |
| ad 100 | Water |

### Example 10

### Cu-Tanmelanin Complex

25 g of copper chloride (CuCl₂.2H₂O) was dissolved in a 1 l of 6% w/v ammonia aqueous solution. To this, 620 ml Tanmelanin of Example 3 was added, followed by stirring at 70-80° C. until there was no unreacted compound. After addition of 100 g of ammonium chloride, the Cr-tanmelanin complex dye was finally salted out, suction filtered and dried.

### Example 11

### Cr-Tanmelanin Complex

25 g of chromium acetate (Cr(CH₃COO)₃.H₂O) was completely dissolved in 800 ml of water while heating. To this solution, 640 ml of Tanmelanin of Example 8 was added, followed by stirring at 70-80° C. and pH 5 to 6.5 until there was no unreacted compound. After addition of 100 g of ammonium chloride, the Cr-tanmelanin complex dye was finally salted out, suction filtered and dried.

### Applicative Example 8

### Leather Dyeing-tanning with Cu-tanmlanin Complex

100 parts of a wet blue bovine box side leather were neutralized in a dyeing drum with 250 ml of water and 0.8 g of Na₂CO₃ at 35° C. during 45 minutes The leather is then washed with 1 l of water at 25° C. After 5 minutes the leather is dyed at 50° C. with 250 ml of water and 16 ml Cu-Tanmelanin complex produced according to Example 10. After 20 minutes 4 ml of a 80% emulsion of a sulfited fish oil were added for fatting and the fatting was continued for 45 minutes Then the bath was acidified with 0.5 ml of a 85% formic acid solution and drumming was continued for 20 minutes Finally the liquor is drained off and the leather was rinsed at 25° C. with 1 l of water. The leather was drained, dried and cured in conventional way to afford a level pastel bordeaux shade.

### Applicative Example 9

### Leather Tanning with Cr-tanmelanin Complex

The same process of Applicative Example 8 was applied with Cr-Tanmelanin complex instead of the Cu-Tanmelanin complex.

### Applicative Example 10

### Textile Dyeing with Cr-Tanmelanin Complex

22.5 mu of defatted wool was wetted in a bath of cold soft water with a ratio of one part wool to 10 parts water for 15 minutes, then drained. The bath of soft water was increased to 25° C. and 3 g/l of triethanolamine was added, obtaining a pH around 9-10. While circulating the bath was warmed to 90° C. in 30 minutes, and maintained at that temperature for an additional 30 minutes The wool was then drained and rinsed with an abundant amount of soft water. At 30° C. 3 g/l of an 80% solution of acetic acid and 4% on the dry fiber weight of Cr-Tanmelanin complex produced according to Example 11 were added to the bath. During a period of 30 minutes, the bath was warmed to 100° C., and kept at that temperature for 20 minutes at a pH of 4. Then 2 g/l of formic acid was added and kept at 100° C. for an additional 15 minutes at a pH of 3.2. Then 5 g/l of lactic acid was added and the bath kept at 100° C. for another 30 minute The bath was then drained and the wool rinsed with abundant soft water and dried. The treated fibers appear light grey/greenish in colour.

### Applicative Example 11

### Textile Dyeing with Native Tanmelanin

1 kg of cotton garments were pretreated in bath of warm water at 40° C. with 4 ml of “Ecowet” (EcoScreen Inc., Ontario, Canada) wetting solution. The bath was added with 25 ml of NaOH 10N and kept at 400 for 20 minutes Then 1.5 l of Tanmelanin of Example 7 was slowly added and the solution was warmed at 60° C. and stirred. The bath was then heated to 70° C. and 0.3 kg of alumn was added and the bath is agitated for ten minutes. The system was then heated to 80° C. and agitated for an additional 45 minutes The cotton rinsed with an abundant amount of soft water and dried. The treated fibers appear beige in colour.

1. A polymer comprising tannin monomers (a), (b) and eumelanic (c) and pyrocatechic (d) monomers according to formula (1): 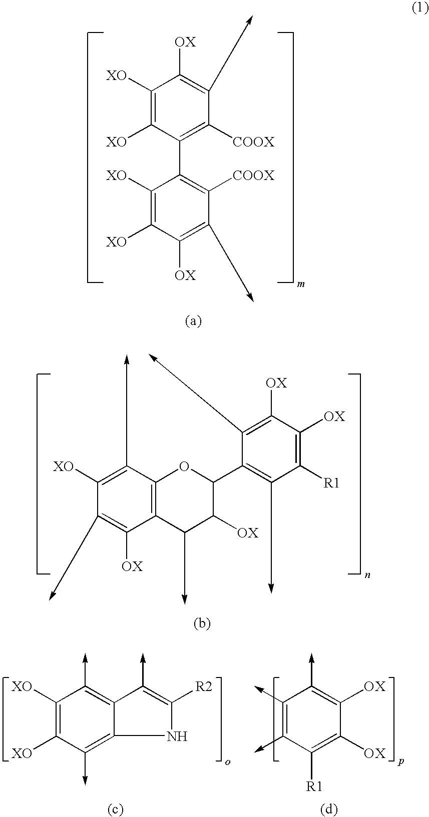
wherein:
→ is a possible intramolecular link between the monomeric units;
m and n are, each independently, a integer between 5 and 30;
o and p are, each independently, a integer between 0 and 30;
R₁ is —OX or —H;
R₂ is —COOX or —H;
each X is independently —H, -G, -M, or a further intramolecular link;
G is a glucosidic, glycosidic or polyolic residue;
M is M₁, 1/y·M₂^{y+}, or 1/z·M₃^{z+};
M₁, is a monovalent cation;
M₂ is a non-chromophoric multivalent cation;
M₃ is a chromophoric multivalent cation;
y and z are integer from 2 to 4 and represents the valence of M₂ and M₃.

2. The polymer according to claim 1 that contains the monomers (a), (b), (c) and (d) with (m+n): (o) comprised between 10:0 and 2:8.

3. The polymer according to claim 1 that contains the monomers (a), (b), (c) and (d) with (m+n): (p) comprised between 10:0 and 4:6.

4. The polymer according to claim 1, wherein the tannin monomers have a tannin precursor that is selected from the group consisting of
ellagitannin of formula (2): 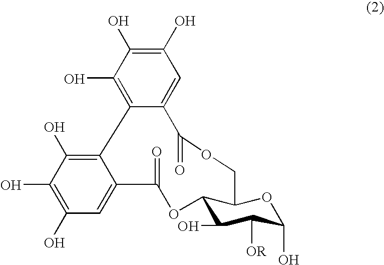
condensed tannin of formula (3): 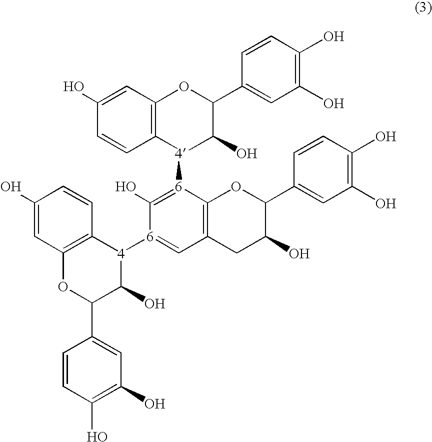
complex tannin of formula (4):

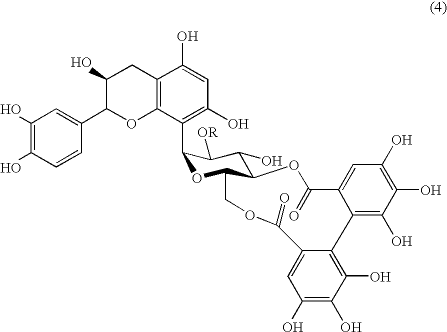

5. The polymer according to claim 4, wherein the tannin precursor is selected between those of formula 2 and 3.

6. The polymer according to claim 1, wherein the eumelanic monomers have an eumelanin precursor (c) that is L-dopa.

7. The polymer according to the claim 1, wherein the pyrocatechic monomers have a pyrocatechic precursor (d) that is pyrocatechin.

8. The polymer according to claim 1, wherein the monovalent cation M₁ is an alkali metal cation or an ammonium cation.

9. The polymer according to claim 1, wherein the non-chromophoric multivalent cation M₂ is a colourless transition metal suitable for metal complex formation.

10. The polymer according to claim 1, wherein the chromophoric multivalent cation M₃ is a coloured transition metal suitable for metal complex formation.

11. The polymer according to claim 1, wherein when X is H the corresponding —OH group may be in a radicalic (—O.) or quinonic (═O) form.

12. A method of preparation of a polymer of formula (1) according to claim 1, which comprises:
(a) formation of an alkaline solution of the precursors by the addition of precursors at 1 and 20% w/v to an aqueous solution containing an amount of an alkali at least stoichiometric with regards to the —COOH and/or —OH groups; and
(b) dispersion of air, or a gas mixture comprising oxygen, into the solution of (a) at a temperature between 0 and 80° C. for a period comprised between 2 and 48 hours to afford tanmelanins in the alkaline salified form.

13. The method according to claim 12, wherein the alkali is NaOH or KOH.

14. The method according to claim 12, wherein the alkaline solution is further added with a catalytic amount of polyvalent cation selected in the group consisting of Fe³⁺, Fe²⁺, Cu⁺, Cu²⁺, Cr⁶⁺, Mn³⁺, Mo³⁺.

15. The method according to claim 12, wherein the alkaline solution is further added, during or at the end of phase (b), with an organic or inorganic peroxide.

16. The method according to claim 15, wherein the peroxide is hydrogen peroxide.

17. The method for the preparation of solid pigments comprising a polymer according to claim 12, said method comprising the co-precipitation of said polymer in the form of alkaline salt (X=M₁) by a polyvalent cation M₂ or M₃ onto a micronized pigments of metal oxide and hydrated oxides selected among TiO₂, Ti(OH)₂, ZnO, Al₂O₃, AlOOH, SnO₂, CeO₄, ZrO₂, FeO, Fe₂O₃, Fe₂O₃.H₂O, FeₓO_{y}, Fe₃O₄, Cr₂O₃.nH₂O, Cr₂O₃, SnO, MnO and mixture thereof.

18. The method according to claim 17 wherein said co-precipitation is carried out with a M₂ polyvalent cation selected from the group consisting of Al, Ca, Zn, Mg, Zn, and Ti.

19. A polymer obtainable with a method according to claim 12.

20. A polymer obtained with the method according to claim 12.

21. A pigment obtained with the method according to claim 17.

22. A topical composition comprising a polymer according to claim 1, wherein X is H or M₁, having bacteriostatic, chelating and antioxidant properties.

23. A cosmetic composition suitable for make-up of face, lips, eyebrows, eyelids and exposed skin (décolleté), said composition comprising a pigment according to claim 17.

24. A cosmetic composition suitable to provide sun protection comprising a pigment according to claim 17.

25. A Tanmelanin-metal complex comprising a polymer according to claim 1 wherein at least one X is M₃.

26. A Tanmelanin-metal complex according to claim 25 wherein the ratio M₁:M₃ is 1:1 or 1:2.

27. A process for the preparation of a tanmelanin-metal complex comprising a reaction of tannin alkali salt (X=M₁) obtained according to claim 12 with a metallizing agents (M₂ or M₃) in a ratio of 1:1 or 1:2.

28. A process for dyeing a substrate comprising the step of applying to said substrate at least one polymer according to claim 1.

29. A process for dyeing a substrate comprising the step of applying to said substrate the tanmelanin-metal complex according to claim 25.

30. A process for dyeing leather comprising the step of applying to said leather the tanmelanin-metal complex according to claim 25.

31. A method for dyeing textile fabric comprising the step of applying to said leather the tanmelanin-metal complex according to claim 25.

32. A substrate dyed by the process according to claim 28.

33. A leather dyed by the process according to claim 30.

34. A textile fabric dyed by the process according to claim 31.

35. The method according to claim 12, further comprising following (a) and (b) by:
(c) partial or complete neutralization by addition of mineral acid or water-soluble organic acid to afford tanmelanin in acidic form.

36. A substrate dyed by the process according to claim 28.
